(19) United States
(12) Patent Application Publication
(10) Pub. No.: US 2025/0101370 A1
(43) Pub. Date: Mar. 27, 2025

(54) METHOD OF YEAST PROPAGATION IN A LOW-COST, TWO-PHASE PROCESS STREAM

(71) Applicants: PETRÓLEO BRASILEIRO S.A. – PETROBRAS, Rio de Janeiro (BR); CENTRO NACIONAL DE PESQUISA EM ENERGIA E MATERIAIS (CNPEM), Campinas (BR)

(72) Inventors: DANUZA NOGUEIRA MOYSES, Rio de Janeiro (BR); VIVIANE MARCOS NASCIMENTO VICENTE, Campinas (BR); ALINE MACHADO DE CASTRO, Rio de Janeiro (BR); CÍNTIA REGINA SARGO, Campinas (BR); FELIPE DE OLIVEIRA BRITO, Rio de Janeiro (BR); ABSAI DA CONCEICAO GOMES, Rio de Janeiro (BR); LUIZ FERNANDO MARTINS BANDEIRA, Rio de Janeiro (BR); CARLOS EDUARDO DRIEMEIER, Campinas (BR); CHRISTIAN ALEJANDRO QUEIPO, Rio de Janeiro (BR); ANA PAULA RODRIGUES TORRES, Rio de Janeiro (BR)

(73) Assignees: PETRÓLEO BRASILEIRO S.A. – PETROBRAS, Rio de Janeiro (BR); CENTRO NACIONAL DE PESQUISA EM ENERGIA E MATERIAIS (CNPEM), Campinas (BR)

(21) Appl. No.: 18/776,031

(22) Filed: Jul. 17, 2024

(30) Foreign Application Priority Data
Sep. 25, 2023 (BR) 1020230196411

(51) Int. Cl. C12N 1/18

(52) U.S. Cl. CPC C12N 1/18

(57) ABSTRACT

The present invention relates to a method of propagating genetically modified yeasts or even any other yeasts that suffer a positive Crabtree effect or any other microorganisms that use sugars for growth (glucose or equivalent glucose), wherein the feeding medium inhibits the optimal exponential growth predicted throughout the process, such a method capable of providing greater process economicity (CAPEX and OPEX), increased productivity and yield (Yx/s) and reduced CAPEX due to greater cell expansion in a single stage of reactor. Particularly, the present invention describes a process for producing second generation ethanol by using different combinations of low-cost streams existing in an integrated 2G or 1G/2G ethanol production plant, combined with the unique batch propagation strategy fed in at least two exponential feeding phases, in just one cycle, with the propagation step occurring in a single reactor.

### FIELD OF APPLICATION

The present invention pertains to the field of biotechnology and bioprocess engineering and chemical processes, more specifically related to microbial propagation to obtain cellular biomass. The present invention provides additional advantages, more specifically, when associated with the propagation for second generation ethanol, but the propagation strategy described herein, in relation to optimizing the cellular biomass production, can be extended to various microorganisms, mainly microorganisms that suffer inhibition of growth by the feeding medium, for example by the Crabtree effect, exemplified here by the yeast Saccharomyces cerevisiae.

Particularly, the present invention relates to a process for producing second generation ethanol from the use of different combinations of low-cost streams existing in a 2G ethanol production plant or in a first generation (1G) ethanol production plant integrated into the 2G production plant, combined with the unique fed-batch propagation strategy, in at least two exponential feeding phases, without feedback control, in just one cycle, with the propagation step occurring in a single reactor. More precisely, the present invention describes a method of propagating genetically modified yeasts or even any other yeasts that suffer a positive Crabtree effect or any other microorganisms that use sugars for growth (glucose or equivalent glucose), wherein the feeding medium inhibits the optimal exponential growth predicted throughout the process, being able to provide greater process savings (CAPEX-Capital Expenditure-capital investment and OPEX Operational Expenditure-Production Cost), increased productivity and cell yield (Yx/s) and CAPEX reduction due to greater cell expansion in a single stage of reactor.

### STATE OF THE ART

The fermentation of lignocellulosic hydrolysates is no longer a bottleneck for 2G ethanol technologies. Currently, there are different options, including commercial ones, of genetically modified yeasts, capable of producing ethanol with high conversion efficiency of the main sugars, such as xylose and glucose (Moysés, 2016). With the use of genetically modified yeasts, it is now possible to achieve conversion rates above 90%, similar to the conversion rates observed in first generation processes, in which wild yeasts are used.

Although the fermentation step is extensively covered in the literature, the propagation step of the aforementioned genetically modified yeasts on an industrial scale is still little discussed. Most processes for ethanol production consider the use and disposal of the yeasts after the hydrolysate fermentation step. This fact is justified by the presence of a high concentration of inhibitors in the medium, such as acetic acid, sugar degradation products and lignin derivatives, which affect cell viability, or further by the presence of solids that hinder the recovery and separation process of the yeasts.

Some ethanol production processes use high inocula, with concentrations greater than 5 g/L of yeast (dry weight), aiming for faster fermentations, which favors the balance between the operational cost and the capital cost with this step and further reduces the risk of microbial contamination. On the other hand, other processes, considering the impact of the propagation step, result in fermentations lasting more than 48 hours, with lower inocula (0.5-1.5 g/L of cells, dry weight). Regardless of the strategy, in general, there is a need to propagate the yeast for each fermentation cycle, which, depending on the concentration of the inoculum in the fermentation, can represent a considerable cost to the process.

Industrial propagation for the production of 1G ethanol, for example, from sugarcane, uses molasses from the sugarcane from the plant itself, plus supplements such as mineral micronutrients, yeast extract, nitrogen sources (for example, urea or ammonium sulfate), phosphorus source (such as phosphates), and eventually peptone, in a single propagation step. To do this, baker's yeast (or similar) is used, acquired on a large scale and from other companies specialized in yeast production. However, there is still no structured market focused on the production of 2G ethanol from genetically modified yeasts or even from any other yeasts that suffer a positive Crabtree effect, and the steps of propagation of said yeasts on a laboratory scale seem to be a responsibility of each plant.

The commercial scale propagation of yeasts dates back to the beginning of the 20^{th} century, between 1905 and 1920, initially occurring in Europe and followed by the United States. At the same time, concerns arose about reducing the cost of the process, which was largely due to the use of molasses as the main source of carbon. Between 1900 and 2009, more than 200 patents were filed on yeast production (Gélinas, 2011). The conventional process takes place in sequential reactors with scale-up, with at least the last stage in the batch strategy fed by molasses supplemented with nitrogen and phosphorus in a C:N ratio between 100:2.5 and 100:5.0; and C:P ratio between 100:0.3 and 100:0.5. The supplements can be fed at the beginning of the batch or throughout the feed along with the sugar stream. Fed-batching is performed with a fixed exponential feeding rate, generally between 0.5 and 0.65 grams of sugar fed per gram of dry yeast per hour, and is often referenced as feeding rate of 0.16-0.18 (gram of sugar fed per gram of yeast per hour) relative to wet yeast dough (assuming 70% moisture). This final step of propagation on an industrial scale occurs during a period of 8 to 16 h, at a temperature of 25 to 35° C., controlled by heat exchangers and a pH between 4 and 6. Different approaches have been advocated over the years in relation to oxygen control, which is essential for the efficiency of this aerobic process, but with the common objective of maintaining excess oxygen throughout growth.

For genetically modified yeasts, most studies are on a laboratory scale, with the propagation step being neglected, often with volumes 3 to 10 times greater than the fermentation itself, for inoculum production in a simple batch, with 2% (m/m) sugars (xylose or glucose) and rich and expensive media, such as YPD (1% yeast extract, 2% peptone and 2% glucose) and its variations. In these propagations, between 3 and 5 g/L of cells are obtained at the end (dry basis) and ethanol production is often also observed. However, the production of ethanol at this step is not desired, both due to the consumption of sugars (which reduces the overall yield of the process) discussed above, and also due to the fact that the aforementioned ethanol is not recovered in the process, after the concentration of the inoculum by centrifugation, since its concentration in the liquid phase does not justify the energy expenditure of the distillation.

Propagation with molasses is an expected alternative, based on experience with wild yeasts; however, in both cases, there is still an undesirable effect on the fermentation of lignocellulosic hydrolysates, which is a possible metabolic adjustment phase (lag phase), due to the greater hostility of lignocellulosic hydrolysates. In this sense, the propagation in the hydrolysate used in fermentation could represent a good process strategy. However, the presence of inhibitors in high concentrations, such as acetic acid in ranges between 4 and 15 g/L, furfural and hydroxymethylfurfural between 100 and 3,000 ppm, phenolic compounds derived from lignin and other minor organic acids, are known to impair the cell viability, more strongly affecting growth capacity (Piotrowski et al., 2014, Cunha et al. 2019, Brandt et al., 2019). Therefore, this ends up being a limiting factor for its direct use in the propagation step, since this step is premised on the generation of quality high inocula to guarantee the efficiency of the fermentation process. Further, another disadvantage of using this hydrolysate stream is its deviation from the main 2G ethanol production process and, consequently, a reduction in the theoretical expected yield of the plant.

2G ethanol production processes that separate the solid fraction from the liquid after the enzymatic hydrolysis (HE) before fermentation, for a solids-free fermentation (as in the example in FIG. 5), may present, in addition to the main stream of hydrolysate rich in sugars (generally varying between 50 and 200 g/L of total reducing sugars, TRS, which includes C5 and C6 sugars), a dilute hydrolysate stream, obtained by washing the filter cake, for example. Washing the filter cake (solid residue from enzymatic hydrolysis) allows the recovery of 10-30% of HE sugars in this stream, which would be lost in the solid. In this sense, the present invention preferably advocates the use of this alternative process stream as a low-cost carbon source option, but not limited to the same, for the yeast propagation process, as detailed below.

In the production of second generation ethanol, whether or not integrated with the production of first generation ethanol (2G or 1G/2G), genetically modified yeasts are used capable of consuming the two main sugars present in the hydrolysate, xylose (C5) and glucose (C6) for maximum use of sugars from lignocellulosic biomass. The various ethanol production processes from hydrolysates rich in C5 and C6 sugars mainly refer to (i) the biomass pre-treatment steps, (ii) enzymatic hydrolysis or (iii) the fermentation itself.

In the propagation step, depending on the inputs used and the fermentation model used, the technology may become economically unfeasible. For example, most co-fermentation processes in the literature indicate fermentation with inocula of 1 to 10 g/L of yeast, generally preceded by growth of the inoculum in rich complex media, such as YP (yeast-peptone extract) medium, with glucose and/or xylose as carbon sources, in addition to other nutrients such as nitrogen and mineral sources. These rich media add considerable cost to the process. Such a strategy of propagating genetically modified yeasts on xylose is used to ensure that the biomass obtained in propagation is only genetically modified yeasts with active and functional xylose metabolism, since only genetically modified yeasts grow on this sugar, unlike wild-type yeasts.

Furthermore, the components of the culture medium are capable of significantly impacting the economic viability of industrial fermentation processes, potentially representing more than 30% of the total production costs of commodities, such as 2G ethanol (Hahn-Hägerdal et al., 2005), being even more critical for processes wherein each fermentation is preceded by a propagation step. In the production of first generation ethanol with sugarcane, this is not a problem, as propagation occurs once, or at most twice, throughout the entire harvest.

Despite the various works already published on co-fermentation in the 2G ethanol process, few studies have focused on how to obtain the inoculum and minimize the costs of the propagation step of genetically modified yeasts or even any other yeasts that suffer a positive Crabtree effect. In this sense, the present invention proposes the use of low-cost streams from the 2G (and/or integrated 1G/2G) ethanol production process itself, not dependent on inputs external to the unit, associated with a strategy that optimizes the use of sugars in the propagation and accelerates fermentation. Such streams are capable of supplying the sugars necessary to reach the cell concentrations usually used in the 2G fermentations.

Document BR 11 2018 015657 4, owned by BETA RENEWABLES S.P.A., published on Aug. 31, 2017, describes a propagation process for genetically modified yeasts. However, said document describes a propagation process in 2 or more cycles, with the need to separate the yeast obtained in one cycle to feed the same in the next cycle, a strategy that entails an additional unitary step in relation to the process described in the present invention, and, consequently, presents greater time and cost for the yeast propagation process. On the other hand, the present invention simplifies this process, allowing propagation to occur in a single operation, divided into at least two substrate feeding phases (which generate two combined growth profiles). This result is obtained by means of a fed-batch strategy carried out in two sequential exponential feeding phases, with distinct exponential profiles. Furthermore, as there is no interruption of the process when passing from the first to the second phase, as both phases occur in a unitary operation, the feeding strategy of the present invention simplifies and significantly reduces the process costs.

In turn, document WO 14072232, owned by DSM IP ASSETS B.V., published on May 15, 2014, describes a process for the aerobic propagation of yeast, in which the yeast is cultivated in a reactor, comprising the steps of: a) filling the reactor with a carbon source and an initial yeast population, b) optionally, cultivating the initial yeast population in the reactor in discontinuous mode, c) measuring the pH in the reactor, d) adding lignocellulosic hydrolysate to the reactor in discontinuous mode with feed, under a rate, in order to adjust the pH in the reactor to a predetermined value, and e) after sufficient propagation, isolating the yeasts obtained in the reactor. The carbon source of step a) of said document may be diluted lignocellulosic hydrolysate, wherein the dilution of the lignocellulosic hydrolysate is provided to reduce the effects of lignocellulosic hydrolysate inhibitory compounds. Although document WO 14072232 describes a propagation process for genetically modified yeasts, with a feeding based on hemicellulosic hydrolysate, with the fed-batch strategy, this feeding merely seeks to maintain the propagation pH at pre-adjusted values. The present invention, in turn, describes an exponential feeding strategy, in at least two phases, which, in addition to maintaining pH at appropriate levels, accelerates the growth of the genetically modified yeasts or even any other yeasts that suffer a positive Crabtree effect. Such a fact can be observed, for example, in the yeast propagation times, which are limited to 30 hours in the present invention and exceed 60 hours in WO 14072232 and, still, with low productivity.

### BRIEF DESCRIPTION OF THE INVENTION

The present invention describes the reuse of low-cost streams within the 1G/2G ethanol production process and the implementation of the described technology, enabling the production of 2G ethanol, and is capable of largely contributing to the reduction of carbon dioxide (CO2) emissions to the environment, when compared to fossil fuels. The excessive release of CO., and further other substances related to the burning of fossil fuels, can have significant negative impacts on both the environment and human health.

Particularly, the present invention describes a method of propagating genetically modified yeasts or even any other yeasts that suffer a positive Crabtree effect, or other microorganisms that use sugars for growth (glucose or equivalent glucose), wherein the feeding medium inhibits the predicted optimal exponential growth throughout the process, exemplified in the present invention for second generation (2G) ethanol, in a low-cost process stream, using at least two exponential feeding phases in a fed-batch, in just one cycle, with the propagation step occurring in a single reactor. The present invention is mainly applied to fermentation technologies, with the objective of obtaining optimized microbial inoculum, thus allowing great cell expansion from alternative carbon sources. More particularly, the present invention refers to integrated 1G/2G ethanol production technology, for propagation and generation of inoculum for co-fermentation, whether with cell recycling or not. However, the feeding strategy, considering the combination of the exponential feeding equations, with two or more growth phases, can be applied to other technologies that involve the propagation of microorganisms.

The propagation step aims at obtaining maximum microbial biomass, corresponding to the conversion of sugars, preferably, to microbial biomass and not to by-products such as ethanol and glycerol. Yeasts such as Saccharomyces cerevisiae, the main genetically modified species usually used for the production of 2G ethanol, are positive Crabtree (DE DEKEN, 1966). This effect makes them produce ethanol through anaerobic metabolism, even in the presence of abundant oxygen, regulated by the concentration of sugars in the medium. Yeasts are capable of growing in both anaerobic fermentation and aerobic propagation. However, the energy balance in aerobic propagation is more advantageous, generating around 10 times more energy, more cellular biomass per consumed sugar, that is, leading to a greater biomass yield-factor Yx/s.

Furthermore, in addition to generating yeast for the subsequent fermentation step, the propagation step has additionally been used as an opportunity to acclimatize the microorganism to the fermentation culture medium. A few studies in the literature indicate that, when the genetically modified yeast is exposed to lignocellulosic hydrolysates since the propagation, it becomes more tolerant to the inhibitors present in these materials. This short-term adaptation of yeast, during the propagation step, can lead to greater ethanol yields and productivity, and a greater capacity to consume xylose in the subsequent co-fermentation step (Nielsen et al., 2015; van Dijk et al., 2021).

Propagation optimization involves the establishment of a low-cost culture medium that provides all the nutrients necessary for the yeast's aerobic metabolism, in addition to a fine adjustment between the supply of sugars and oxygen for growth and consumption of these by the growing yeast population. The presence of excess sugars can lead to changes in metabolism and ethanol production, while the rapid consumption of sugars and aerobic growth of the population can lead to deprivation of oxygen and sugars, also hindering the microbial growth. Ideally, the best growth would be achieved with an increasing feeding of sugars and an increasing supply of oxygen, following cell growth rates, so that the sugar-free concentration remains low (<0.1% TRS-m/m) in the medium, avoiding the Crabtree effect. In practice, this real-time control and monitoring of the microbial growth and adjustment of the feeding flow rate (with feedback control) are not very feasible, and are also influenced by other factors, such as nutrient scarcity, presence of inhibitors in low-cost sugar streams, and cellular signaling, for example, regarding cell density. All these factors and others, in synergy or not, act by reducing the cell growth, with losses to the process, deviating from the exponential growth curve expected by the models used to define the process parameters, such as, for example, the speed of sugar feeding.

To overcome the discussed process difficulties, the present invention describes a fed-batch strategy without feedback control, with exponential feeding of sugars (based on equation 1) in at least two phases (but not limited thereto), which allows in a single stage of reactor the output of low biomass inocula (e.g. the equivalent of 0.1-0.3 g/L of cells relative to the final volume, dry weight, at the start of the fed-batch) and the range of high cell concentration (>15 g/L cells, on a dry basis), using low-cost process streams, such as dilute lignocellulosic hydrolysate streams. Particularly, the fed-batch feeding strategy, in at least two phases, increases productivity and reduces propagation time to obtain inocula above 10 g/L (between 15 and 30 h).

The strategy combines the fastest specific growth phase with an exponential feeding, calculated by the equation considering a high μₛₑₜ (specific growth rate adopted) in the first hours of the process (6-9 hours) (time required to reach cell concentration close to 5 g/L), and a phase of slower specific growth from 6-9 hours, until the end of the process (15-30 hours), considering a lower μₛₑₜ, with a relatively high initial cell concentration, for example, between 3 and 10 g/L in total volume. This strategy that combines two distinct exponential feeding phases is exemplified in FIG. 1, called strategy #3, also showing the expected curves for strategy #1, if the exponential feeding curve were in a single phase considering the high μₛₑₜ throughout the feeding, and strategy #2, considering a single feeding phase with a low μₛₑₜ. Strategy #3 allowed the achievement of a surprising propagation result, that is, the expansion of more than 100 times in biomass in relation to the initial cell concentration, starting from inocula with low biomass concentration (0.1-0.5 g/L, considering the final propagation volume) and reaching final concentrations close to 30 g/L of cells (dry basis) on a pilot scale.

Furthermore, based on the strategy described in the present invention, it was possible to obtain inoculum for a fermentation process with minimizing the costs of the propagation step of genetically modified yeasts (GMO) or even any other yeasts that suffer a positive Crabtree effect, or other microorganisms that use sugars for growth (glucose or equivalent glucose), wherein the feeding medium inhibits the optimal exponential growth predicted throughout the process, with optimization of the use of sugars in propagation, reduction of the production of by-products such as ethanol and glycerol, and acceleration of the fermentation due to the exposure of microorganisms to lignocellulosic hydrolysates. This last advantage, that is, the advantage of accelerating fermentation, occurs more specifically when associated with the production of 2G ethanol, by GMO yeasts, such as Saccharomyces cerevisiae, as exemplified herein.

### BRIEF DESCRIPTION OF FIGURES

FIG. 1 an is exemplary graphical representation of the expected exponential feeding curves for strategy #1 (light gray color, followed by dashed lines), strategy #2 (dark gray color, dashed line followed by solid lines) and strategy #3 (by combining the two strategies, encompassing the solid line phases of the two previous strategies) for the propagation of genetically modified yeasts.

FIG. 2 is a graphic representation of the concentrated hydrolysate stream fermentation when using genetically modified yeast grown in propagation media containing diluted hydrolysate stream, diluted molasses or a mixture of the two.

FIG. 3 is a graphical representation of experimental data on the variation in cell concentration in propagations of genetically modified yeasts in a fed-batch bioreactor conducted in a single phase (exponential feeding strategy #1 and exponential feeding strategy #2), using a diluted stream of hydrolysate combined with molasses, supplemented with low concentrations of yeast extract and ammonium sulfate.

FIG. 4 is a graphical representation of experimental data regarding Strategy #3 of fed-batch propagation without feedback control with exponential carbon source feeding profile in two pre-defined feeding phases using genetically modified yeasts (yeasts A and B) and a diluted stream of hydrolysate combined with molasses, supplemented with low concentrations of yeast extract and ammonium sulfate. The cell concentration is relative to the final reaction volume. In all cases in the feeding, a stream between 60 and 80 g/L of sugars was provided, except for the A yeast in pilot scale identified as “HH”, in which a more concentrated feeding stream was applied, ˜150 g/L of sugars. Each curve corresponds to an experimental test with the aforementioned yeast (A or B) on the aforementioned scale (pilot or laboratory) indicated in the key, and are not replicas, since the tests used hydrolysates from different runs.

FIG. 5 is a schematic of the 2G ethanol production process, with an example of the step of recovering the diluted hydrolysate stream.

### DETAILED DESCRIPTION OF THE INVENTION

The present invention refers to a propagation process for generating inoculum for fermentation, exemplified for the production of second generation ethanol by using different combinations of low-cost streams existing in an integrated 2G or 1G/2G ethanol production plant, combined with the unique batch propagation strategy fed in at least two exponential feeding phases, in just one cycle, with the propagation step occurring in a single reactor. The present invention provides additional advantages, more specifically, when associated with the propagation for 2G ethanol, as it acclimatizes the microorganism and accelerates the subsequent fermentation step. However, the propagation strategy described herein, in relation to optimizing the production of cellular biomass, can be extended to several microorganisms, mainly microorganisms that suffer growth inhibition by the feeding medium, for example by the Crabtree effect, exemplified herein by the genetically modified Saccharomyces cerevisiae yeasts. Particularly, the present invention describes a method of propagating genetically modified yeasts or even any other yeasts that suffer a positive Crabtree effect, or other microorganisms that use sugars for growth (glucose or equivalent glucose), in which the feeding medium inhibits the optimal exponential growth predicted throughout the process, capable of providing greater process savings (CAPEX and OPEX), increased productivity and yield in microbial biomass (Yx/s) and reduced CAPEX due to greater cell expansion in a single stage of reactor.

In the second generation (2G) ethanol plant, preferably a 1G/2G integrated plant, the main sources of carbon available locally for propagation would be: the lignocellulosic hydrolysate (rich in xylose and glucose), the molasses and sugarcane juice of the 1G plant (rich in sucrose, glucose and fructose), and preferably a diluted stream of hydrolysate generated, for example, by washing the filter cake, in case of solid-liquid separation after the enzymatic hydrolysis step (before the fermentation). In relation to cost, this last stream would be the most advantageous, as its use in the fermentation process would require an extra step of sugar concentration and, on the other hand, lower concentrations of sugars are desired in the propagation. Another locally available process stream as a nutrient source for propagation is the yeast extract, obtained by periodically purging yeasts from the 1G process or, eventually, by yeasts that may be discarded after the 2G fermentation, followed by an autolysis process. Both molasses and yeast extract are products eventually sold by 1G ethanol plants, and can also be purchased as inputs for the present invention.

The present invention demonstrates, in an unprecedented way, that the use of hydrolysate diluted in the propagation, as long as the minimum tested concentration of hydrolysate is guaranteed (8-16% in relation to the hydrolysate from the fermentation step) as the only source of carbon or as a complementary source, generates a positive effect of acclimatization and improvement of the fermentation. In addition, the use of diluted hydrolysate in the propagation of the present invention also indicates the possibility of process flexibility, being able to have the stream with a greater or lesser proportion of molasses in relation to the hydrolysate one, preferably maintaining a hydrolysate concentration greater than 8% in relation to the hydrolysate from the fermentation step, and in order to guarantee the concentration of “equivalent glucose” (sucrose, glucose and fructose) between 40 and 140 g/L.

It is worth emphasizing that, depending on the configuration of the 2G ethanol production process, more specifically the pre-treatment and neutralization steps, the supplementation of the propagation step with a nitrogen source, such as, for example, urea or ammonium sulfate, may or may not be necessary. In processes, such as, for example, dilute acid or hydrothermal pre-treatment, in which the neutralization step or the pH adjustment is carried out with ammonium hydroxide, a nitrogen source is supplied to the hydrolysate, further reducing the cost of the propagation step. In this case, the propagation step would depend only on the hydrolysate stream (with or without added molasses) and low concentrations of yeast extract (2-6 g/L), which could be obtained locally, coming from the cells from the purge of the fermentation process.

The present invention brings a simplified and very efficient methodology that can be applied, including industrially, capable of maintaining a high growth rate of genetically modified yeasts or even any other yeasts that suffer a positive Crabtree effect and minimizing the production of ethanol and other possible by-products, unwanted at this step. The methodology allows, in a single reactor, to go from low inoculum concentrations and reach high cell density, through the fed-batch propagation strategy without feedback control, with an exponential supply profile of carbon and nutrient sources, with at least two pre-defined feeding phases.

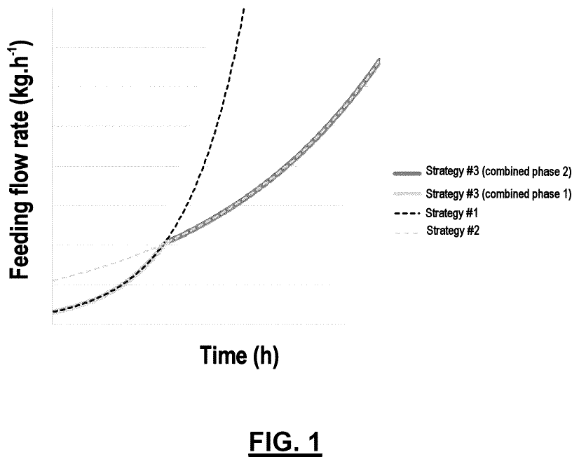

The propagation with a low initial inoculum concentration presents a higher specific growth rate (in the presence of hydrolysate), while, after reaching a higher cell concentration (3 to 8 m·s·m·s·kg⁻¹ or 3 to 8 grams in weight of dry cells per kilo) and greater presence of inhibitors that make up the hydrolysate, the specific growth rate is considerably reduced, leading to the accumulation of sugars and ethanol production. The innovative strategy consists of combining the fastest specific growth phase with an exponential feed calculated by the equation, considering a high μₛₑₜ (adopted specific growth speed) in the first hours of the process (6-9 hours) (time required to reach cell concentration close to 5 g/L) and a slower specific growth phase from 6-9 hours, until the end of the process (15-30 hours), considering a lower μₛₑₜ. This therefore generates a growth with two distinct phases of exponential feeding, according to the feeding flow rate exemplified below (FIG. 1), which was applied both on a bench scale (1 to 3 L) and in a pilot reactor (up to 120 L). Strategy #3, which combines the two distinct phases of exponential feeding, allowed the expansion of more than 100× in initial cellular biomass, starting from inocula with low biomass concentration (0.1-0.5 g/L, considering the final propagation volume) and reaching final concentrations close to 30 g/L of cells (dry basis), on a pilot scale, between 15 and 30 hours, demonstrating high cell yield and high productivity.

The exponential feeding profile F(t) of the two or more phases can be automatically controlled by the supervisory system of the bioreactor system based on equation 1 (Enfors and Häggström, 1998), adapted to the preferred sugar consumption profile, wherein, in equation 1, the sugars present in the feeding stream that are preferentially consumed by the microorganism are considered equivalent glucose. The feeding being started at time 0 h of the propagation and, after 6-9 hours of propagation, the value of the adopted specific growth speed (μₛₑₜ) is changed manually (but not limited thereto), in order to avoid overfeeding the carbon and nutrients source, and consequently, reduce the production of unwanted metabolites, thus ensuring greater biomass production.

$$F ( t ) = ( \frac{{μ}_{set}}{{Y}_{X / S}} ) \frac{{C}_{X 0} {M}_{0}}{{C}_{S}} {e}^{{μ}_{set} t} \qquad Equation ( 1 )$$

where:
F(t)=fresh medium feeding flow rate (kg/h);
μₛₑₜ=adopted specific growth speed (h⁻¹);
Y_{X/S}=cell yield (m·s./g equivalent glucose);
C_{X0}=initial cell concentration (m·s./kg);
M₀=mass at the beginning of feeding (kg);
t=time (h);
C_{S0}=equivalent glucose concentration in the feeding medium (g equivalent glucose/kg)

$$equivalent glucose = {C}_{glucose} + {C}_{fructose} + {C}_{sucrose} / 0.95$$

Equation 1 takes into account the initial inoculum concentration, the medium mass, the specific growth rate and cell yield expected for each phase, and the concentration of sugars in the feeding stream, to determine the exponential variation of the substrate stream feeding flow rate for each phase, thus allowing the automatic adjustment of the feeding flow rate following the exponential growth predicted for each phase. The present invention exemplifies the combination of at least two phases, with gains in yield and productivity, but is not limited to just two exponential phases. A particularity of the present invention in relation to the use of equation 1 (Enfors and Häggström, 1998), is the fact that only glucose, fructose and sucrose are considered as substrate concentration in the feeding medium, grouped as equivalent glucose, instead of the total sugars present in the medium. This adaptation of the equation was carried out, considering the profile of sugar consumption by the genetically modified yeasts, since, despite being able to consume the xylose present in the hydrolysate, the consumption of this sugar is delayed in the presence of glucose, due to competition from its entry into the cell, as it depends on transporters with greater affinity for glucose (Subtil and Boles, 2012). The present invention demonstrates the application of equation 1 adapted to the sugars preferentially consumed by yeast, example, sucrose, glucose and fructose, present in molasses or hydrolysate, or in a combination of the two in the feeding stream. When applying this methodology to other microorganisms or other preferred sugar streams, it is necessary to adjust the equation considering the sugars preferentially consumed by the microorganism as equivalent glucose.

Phase 1—first 6-9 hours of testing. Low initial cell concentration (C_{X0}˜0.2-1.5 m·s./kg, in the initial volume) and a cell growth speed (μₛₑₜ between 0.26 and 0.32 h⁻¹) close to the maximum growth specific rate (μₘₐₓ) is considered, thus guaranteeing a feeding flow rate, providing a relatively high amount of sugar (g of added sugar per g of existing biomass), in order to reach ˜3-8 m·s./kg;

Phase 2—from 6-9 hours to 15-30 hours. At this phase, a feeding flow rate providing a relatively smaller amount of sugars (g of added sugar per g of existing biomass) was imposed, by decreasing the growth rate adopted in the equation (μₛₑₜ between 0.06 and 0.12 h⁻¹), in order to limit the supply of substrate and, consequently, the unwanted production of ethanol.

This technology allows the reduction of investment in equipment in the propagation step (CAPEX), reduces the normalized operating cost for propagation (OPEX), reduces the number of steps and scales of reactors, reduces the operational costs by improving productivity and cell efficiency (Y_{X/S}), as well as the use of low-cost streams available in the unit. Furthermore, the preferential use of the hydrolysate stream allows a fermentation with a higher speed of initial consumption of the sugars present in the hydrolysate, leading to an increased productivity also in the fermentation step.

### Steps in the Propagation Process

In the 2G ethanol production process, in a plant integrated with the sugar or 1G ethanol plant or not, the biomass (1) is sent to the biomass pre-treatment step (2), preferably, but not limited to the acidic pre-treatment of biomass with diluted sulfuric acid or hydrothermal pre-treatment (self-catalytic due to the organic acids in the biomass itself), which can be in one or more stages, also with steam explosion. This biomass is preferably sugarcane bagasse, energy cane (whole or bagasse) and straw, but extendable to other lignocellulosic biomasses.

The pre-treated biomass (3) goes through a neutralization step (5) with a basic chemical compound (4), preferably with ammonium hydroxide, ammonia, sodium or potassium hydroxide, but not limited to these bases. However, the first two bring benefits to the propagation step (17) by adding nitrogen to the process stream. After the neutralization step (5) until a pH close to the ideal for the enzymes used in enzymatic hydrolysis (7), the pre-treated and neutralized biomass (6) undergoes hydrolysis by the action of enzymes, mainly cellulases. At the end of this step, the stream containing a mixture of hydrolysate and undigested fiber (insoluble solid) (8) is directed to solid-liquid separation (9 and 12), preferably by filtration. This step generates the main stream of hydrolysate free of insoluble solids, rich in sugars and with a strong presence of inhibitors (10) and the insoluble solid corresponding to undigested fiber, rich in lignin (also called enzymatic hydrolysis residue). In this example, the filter cake (12) is then washed with water (11), generating a stream of diluted hydrolysate. At the end of the solid-liquid separation, 3 streams are obtained: i) lignocellulosic hydrolysate rich in sugars (10), ii) dilute hydrolysate (13) and iii) enzymatic hydrolysis residue (solid fraction—14)

Parallel to the 2G ethanol process, in the 1G ethanol process, there is the production of molasses (15), which can be added to the sugarcane juice treated for fermentation to produce 1G ethanol. In some cases, yeast extract (16) is produced by purging yeasts from the 1G fermentation, since, in the 1G process, it is constantly necessary to cut to reduce the inoculum for the next fermentation cycle. In fermentation to produce 1G ethanol, 90% of the yeasts are returned to the next fermentation cycle and 10% of the yeasts are purged in each cycle, corresponding to the increase in inoculum due to the propagation of the yeasts in the anaerobic fermentation. The yeast extract (16) can also be considered as a lower cost stream in 1G, 2G or integrated 1G/2G ethanol plants, and can eventually also be obtained by recovering genetically modified yeasts (preferably for internal and non-commercial use). The yeast extract is rich in nutrients, mainly nitrogen, as well as essential micronutrients necessary for efficient propagation.

The present invention specifically addresses to the propagation step (17), in this case, following the step of enzymatic hydrolysis of the biomass, preferably taking advantage of the diluted hydrolysate stream (13) with 20-70 g/L of sugars, for example 40 g/L TRS (between 30 and 50% xylose and 40 and 80% glucose), added or not with molasses (for example in a 1:1 ratio of TRS), supplemented with yeast extract (16) in low concentration (2-6 g/L). Alternatively, the main sugar-rich hydrolysate stream can replace the diluted stream, for example in processes wherein there is no solid: liquid separation. This can be diluted to adjust the concentration of inhibitors and final concentration of sugars desired in the feeding stream (between 40 and 150 g/L of TRS, but not limited thereto, preferably between 70 and 120 g/L), as the invention proves the advantage the presence of hydrolysate in the propagation, even if diluted, since it shortens the lag phase of the fermentation that follows the same.

Propagation takes place in a fed-batch regime with temperatures between 25 and 35° C., pH controlled between 4 and 6, through the automatic addition, preferably of an aqueous solution of NH₄OH (10-30% v/v) and H₂SO₄ (0.5-2 M), but not limited thereto, since other bases and acids can also be used for pH adjustment at concentrations other than those indicated. The dissolved oxygen (DO) concentration was maintained above 30% of saturation through the automatic cascade, with adjustment of the stirring speed (between 100 and 900 rpm) and air flow rate (between 0.5 and 4.0 L/min), although not limited to this condition of aeration and stirring, since the objective of the present invention was not to evaluate the effect of oxygenation. The initial medium can be composed, for example, of 2-6 g/L of yeast extract and 1-5 g/L of ammonium sulfate, containing all the required supplementation, or also added directly to the feeding stream. The feeding medium is preferably composed of 25-100% diluted hydrolysate (for example 45-55 g/kg equivalent glucose) and diluted molasses, in order to obtain preferably, but not limited thereto, a medium with ˜60-120 g/kg of equivalent glucose.

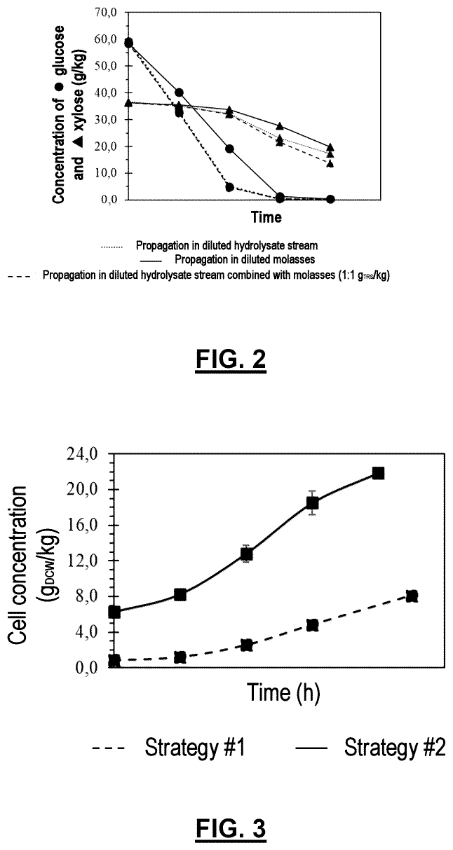

The propagation step (17) generates the inoculum (18) at a high cell concentration (15-30 g/L of cells), which can be used directly in the fermentation step (19), or in the case of fermentations that use highest concentration of inoculum (21), go through a simple concentration step (20), for example, by centrifuges. In the fermentation step (19) for the production of 2G ethanol, the quality inoculum generated in the present invention converts the sugars in the hydrolysate into ethanol, without a long lag phase, since the propagation step is also responsible for an acclimatization of the yeast-Saccharomyces cerevisiae—to the hydrolysate and its inhibitors (shown in FIG. 2).

Propagation with a mixture of diluted hydrolysate (13—diluted stream or 10—main stream hydrolysate after dilution), molasses (15) and yeast extract (16) in low concentration, proved capable of being carried out in at least at least 3 propagation scale-up sequences, without compromising fermentation performance, allowing scale-up from, for example, 1:50 to up to 1:100 in the reactors. Depending on the pre-treatment chosen, as well as the base (4) of choice for neutralization (5), it may be necessary to supplement with external nitrogen sources, such as, for example, ammonium sulfate, even at low concentrations 1-5 g/L. However, when the pre-treatment is preferably a dilute acid pre-treatment, for example with 0.5-3% (m/m) sulfuric acid, and the base of choice for neutralization of biomass and pH control of hydrolysis enzymatic and propagation is ammonium hydroxide, wherein the biomass is, specifically, sugarcane bagasse or energy cane, the supplementation with other sources of nitrogen is not necessary.

After the fermentation step (19), the fermented stream, also called wine (22) is subjected to a solid: liquid separation process (23), obtaining the cell-rich stream (24) and the deyeasted wine (27). The cell-rich stream (24) undergoes an autolysis process (25), and the stream (26) can be incorporated into the yeast extract stream (16). The deyeasted wine (27) is sent to the distillation step (28), where two streams are obtained: hydrated ethanol (30) and vinasse (29). Hydrated ethanol can go through a dehydration process (31), for example, using a molecular sieve, in order to produce anhydrous ethanol (32).

### Results

Studies in stirred flasks have shown that the diluted hydrolysate stream is a carbon source that allows the propagation of genetically modified yeasts or even any other yeasts that undergo a positive Crabtree effect capable of consuming glucose and xylose present in the hydrolysate, without the addition of nutrients. However, low concentrations of yeast extract (2 to 6 g/L) significantly favor the cell growth of the genetically modified yeasts or even any other yeasts that suffer a positive Crabtree effect. Two genetically modified yeasts from different origins (A and B) were evaluated, which responded in a similar way, with considerable improvement in growth, with the supplementation of low concentrations of yeast extract. As an example, when using a diluted stream of hydrolysate (but not limited thereto), with or without different proportions of molasses, totaling 15 to 25 g_{TRS}/kg, it was possible to achieve cell concentrations in the ranges of 3 to 5.5 m·s./kg with the addition of yeast extract, while without supplementation between 1.5 and 2.5 m·s./kg was obtained in stirred flask tests.

The results of using the diluted hydrolysate stream, in combination with molasses in different proportions, demonstrated that the propagation, even in conditions with inhibitors corresponding to less than 10% of the concentration of inhibitors present in the main hydrolysate stream used for fermentation, is capable of generating an acclimatization effect for yeast (a phenomenon also known as short-term adaptation) and reducing the lag phase of sugar consumption in the fermentation step for ethanol production (FIG. 2). In FIG. 2, in order to illustrate this phenomenon, there is presented an example of comparison of the initial fermentation times of the lignocellulosic hydrolysate, using a genetically modified yeast grown in propagation: only with diluted molasses (solid line), or only with diluted hydrolysate (dotted line) or with diluted hydrolysate plus molasses (dashed line). This example demonstrates that the propagation step can impact the fermentation performance, especially in the first hours. The use of a diluted stream of hydrolysate (with or without molasses added) in the propagation of the genetically modified yeasts was capable of generating an acclimatization effect thereof in face of the xylose and inhibitors present in the main stream of hydrolysate used for fermentation. As observed, this acclimation was able to reduce the lag phase of sugar consumption in the ethanol production of the fermentation step, when compared to fermentations conducted with yeast grown in media without the presence of hydrolysate streams (in FIG. 2, diluted molasses).

In comparison with the strategy advocated in the present invention, fed-batch without feedback control, with two exponential phases, feeding two different propagation conditions were evaluated in a bioreactor in fed-batch mode in a single phase (FIG. 3: Strategy #1) starting with a low inoculum (between 0.8 and 1.5 m·s./kg) and imposing a cell growth rate close to the maximum growth speed of genetically modified yeasts (μₛₑₜ between 0.26 and 0.32 h⁻¹);

Strategy #2 (FIG. 3): starting with a high inoculum (between 5 and 7 m·s./kg) and imposing a relatively low cell growth rate (μₛₑₜ between 0.06 and 0.12 h⁻¹), in order to limit the supply of sugars. Strategies #1 and #2 had the same operating conditions and differed only in the carbon source feeding curve and the initial concentration of the inoculum, in accordance with the respective strategies #1 and #2 presented in FIG. 1.

Strategy #1 demonstrated an expansion of 12-14× in cell mass, whereas in strategy #2, this expansion was only 5-7× in dry cell mass. In terms of cell yield, strategy #1 allows lower yields, in the range of 0.25-0.3 m·s./g_{TRS} supplied, whereas in strategy #2, the yields were slightly higher, between 0.32-0.38 m·s./g_{TRS}.

The propagation strategy, object of this invention, was developed with the aim of allowing a process with high cell expansion, starting from a low inoculum, and guaranteeing a high cell yield, preferably in a single stage of reactor, to better use of the sugar streams from this process. In this sense, the innovative strategy, here called strategy #3, presents 2 distinct feeding regime phases: Phase 1, which follows the feeding evaluated in strategy #1 during the initial 6 to 9 hours; and Phase 2, which follows strategy #2, until completing 15 to 30 hours of process (FIG. 1).

Strategy #3, object of the present invention, demonstrated a high expansion in yeast mass of more than 45× on a laboratory scale (1 L) and between 50 and 117× on a pilot scale (80-120 L), with cell yields in the range of 0.2-0.55 m·s./g_{TRS}. This result is superior to that obtained by strategies #1 and #2, since strategy #3 combines the growth phases wherein μₘₐₓ is closer to μₛₑₜ, in each case. The increase in scale not only confirmed the success of the strategy, but also showed even higher yields. The best results obtained with the increased scale must be related to 2 operational factors: 1) better control of the feeding at very low flow rates and 2) less effect of sampling in reducing the reactor's resident biomass. In addition to these factors, others inherent to the increase in scale, such as, for example, the greater solubilization of oxygen promoted by the greater pressure applied to the reactors, cannot be discarded. It is worth emphasizing that, in the reported experiments, dissolved oxygen was maintained above 30% of saturation, by automatic cascade control (with adjustment of stirring speed and air flow rate), not being a variable studied in the present invention.

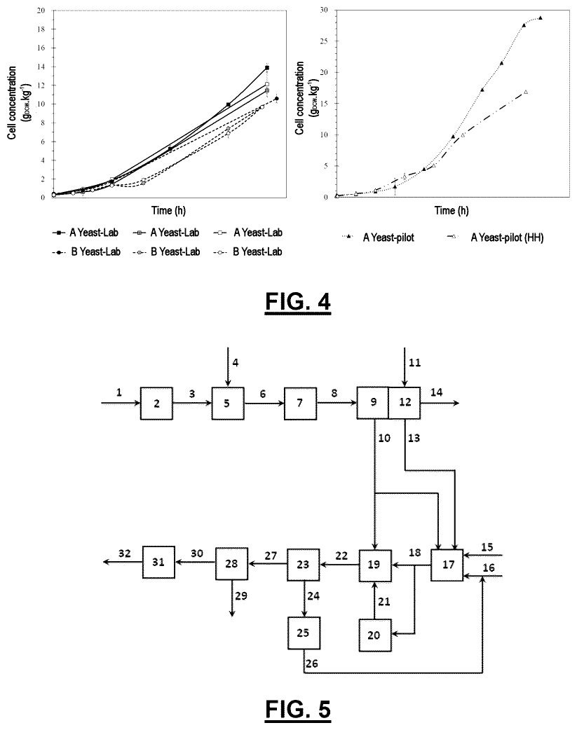

In FIG. 4, examples of the cell growth kinetics on a laboratory scale are presented, with two genetically modified yeasts (A and B), from different origins, capable of consuming xylose, and the kinetics on a pilot scale for one of the yeasts (A). The parameters of the feeding curves were studied and defined based on the rates obtained with yeast A. However, it was observed that even without specific adjustments, the same strategy, when adopted for another genetically modified yeast (B), in the laboratory, resulted in satisfactory growth, showing robustness of the process with different strains. But preferably, it is recommended to adjust the parameters of the equations to the specific rates of each strain, in order to obtain the best possible yields.

In table 1, there is presented an example of the embodiment of the composition of a sugarcane lignocellulosic hydrolysate stream and a molasses stream, but not limited to this composition, since the process proved to be flexible and capable of obtaining similar results, with lignocellulosic hydrolysate streams from different pre-treatment technologies and different lignocellulosic biomasses (such as sugarcane bagasse, straw, energy cane, both with hydrothermal pre-treatments and dilute acids, but not limited thereto).

TABLE 1
Example of the composition of a sugarcane lignocellulosic
hydrolysate and the molasses stream. Both before
being diluted for the propagation step.

| Concentration | Concentrated | |
|---|---|---|
| (g/kg) | hydrolysate stream | Molasses stream |
| Arabinose | 1.0-4.0 | — |
| Xylose | 40.0-50.0 | — |
| Glucose | 55.0-70.0 | 55.0-80.0 |
| Fructose | — | 55.0-65.0 |
| Sucrose | — | 250.0-400.0 |
| Acetic Acid | 6.0-8.0 | 0.5-1.5 |
| Levulinic Acid | 0.0-0.2 | — |
| HMF | 0.1-0.4 | — |
| Furfural | 0.2-0.4 | — |

Therefore, the present invention is described in terms of its preferred embodiment, it being clear that modifications can be made to the matter described herein, such modifications still being encompassed by the set of claims comprising this description.

### BIBLIOGRAPHIC REFERENCES

De Dekken, R. H. (1966). The Crabtree Effect: A Regulatory System in Yeast 149-156.
Enfors, S. O., Häggström, L. (1998). Bioprocess technology: fundamentals and applications. Royal Institute of Technology, Stockholm.
Hahn-Hägerdal, B., Karhumaa, K., Larsson, C. U., Gorwa-Grauslund, M., Gorgens, J., & Van Zyl, W. H. (2005). Role of cultivation media in the development of yeast strains for large scale industrial use. Microbial cell factories, 4 (1), 1-16.
van Dijk, M., Rugbjerg, P., Nygård, Y., & Olsson, L. (2021). RNA sequencing reveals metabolic and regulatory changes leading to more robust fermentation performance during short-term adaptation of Saccharomyces cerevisiae to lignocellulosic inhibitors. Biotechnology for biofuels, 14 (1), 1-16.

Nielsen, F., Tomás-Pejó, E., Olsson, L., & Wallberg, O. (2015). Short-term adaptation during propagation improves the performance of xylose-fermenting Saccharomyces cerevisiae in simultaneous saccharification and co-fermentation. Biotechnology for Biofuels, 8 (1), 1-15.
Moysés, D. N., Reis, V. C. B., Almeida, J. R. M. D., Moraes, L. M. P. D., & Torres, F. A. G. (2016). Xylose fermentation by Saccharomyces cerevisiae: challenges and prospects. International Journal of Molecular Sciences, 17 (3), 207.
Gélinas, P. (2010). Mapping early patents on baker's yeast manufacture. Comprehensive Reviews in Food Science and Food Safety, 9, 483-497.
Piotrowski, J. S., Zhang, Y., Bates, D. M., Keating, D. H., Sato, T. K., Ong, I. M., & Landick, R. (2014). Death by a thousand cuts: the challenges and diverse landscape of lignocellulosic hydrolysate inhibitors. Frontiers in microbiology, 5, 90.
Cunha, J. T., Romani, A., Costa, C. E., Sá-Correia, I., Domingues, L. (2019). Molecular and physiological basis of Saccharomyces cerevisiae tolerance to adverse lignocellulose-based process conditions. Applied microbiology and biotechnology, 103, 159-175.
Brandt, B. A., Jansen, T., Görgens, J. F., & van Zyl, W. H. (2019). Overcoming lignocellulose-derived microbial inhibitors: advancing the Saccharomyces cerevisiae resistance toolbox. Biofuels, Bioproducts and Biorefining, 13 (6), 1520-1536.

1. A method of propagating microorganisms to produce inoculum for fermentation, comprising the step of inoculum growth, wherein said method has at least two feeding phases, with an exponential feeding profile F(t), in fed-batch, phases 1 and 2, without feedback control, in a single reactor, wherein:
Phase 1 occurs in the first 6 to 9 hours of testing, with a low initial cell concentration in the initial volume and supply of a relatively high amount of sugar (g of added sugar per g of existing biomass), for a cell growth speed close to that of maximum specific growth speed; and
Phase 2 occurs from 6 to 9 hours to 15 to 30 hours, with an initial cell concentration between 3 and 10 g/L in total volume and supply of a relatively low amount of sugar (g of added sugar per g of existing biomass), imposing a lower growth rate.

2. The method according to claim 1, wherein the exponential feeding profile F(t), of the two or more phases, is automatically controlled by the supervisory system of the bioreactor system based on equation 1 below, in which equivalent glucose is considered to be the sugars present in the feeding stream that are preferentially consumed by the microorganism:
$$F ( t ) = ( \frac{{μ}_{set}}{{Y}_{X / S}} ) \frac{{C}_{X 0} {M}_{0}}{{C}_{S}} {e}^{{μ}_{set} t} \qquad Equation ( 1 )$$
where:
F(t)=fresh medium feeding flow rate (kg/h);
μₛₑₜ=adopted specific growth rate (h⁻¹);
Y_{X/S}=cell yield (m·s·/g equivalent glucose);
C_{X0}=initial cell concentration (m·s·/kg);
M₀=mass at the beginning of feeding (kg);
t=time (h);
C_{S0}=equivalent glucose concentration in the feeding medium (g equivalent glucose/kg);
in the application example, the equivalent glucose corresponds to the sum of glucose, fructose and sucrose/0.95.

3. The method according to claim 1, wherein the yeast propagation occurs in a fed-batch with temperatures between 25 and 35° C. and pH between 4 and 6;
wherein the pH of the medium is maintained through the addition of acid or base solutions, preferably an aqueous solution of NH₄OH (10-30% v/v) and H₂SO₄ (0.5-2 M); and
wherein the concentration of dissolved oxygen in the medium is maintained above 30% of saturation through automatic cascade control with adjustment of the stirring speed (between 100 and 900 rpm) and air flow rate (between 0.5 and 4.0 L·min⁻¹).

4. The method according to claim 1, wherein the minimum concentration of hydrolysate used in the propagation process is 8-16% in relation to the hydrolysate from the fermentation step, as the only source of carbon or as a complementary source.

5. The method according to claim 1, wherein the lignocellulosic hydrolysate stream is a diluted lignocellulosic hydrolysate stream, having a minimum concentration of 8-16% in relation to the hydrolysate from the fermentation step, for acclimatization effect.

6. The method according to claim 1, wherein the lignocellulosic hydrolysate stream is provided to the reactor, optionally, in combination with a molasses stream in different proportions, preferably for a hydrolysate concentration greater than 8% in relation to the hydrolysate from the fermentation step, and in order to guarantee the equivalent glucose concentration (sucrose, glucose and fructose) between 40 and 140 g/L.

7. The method according to claim 1, wherein the initial cell concentration of the propagation process corresponds to approximately 0.2-1.5 m·s·/kg in the initial volume.

8. The method according to claim 1, wherein the adopted specific cell growth rate (μₛₑₜ) in phase 1 is between 0.26 and 0.32 h⁻¹ and the adopted specific cell growth rate (μₛₑₜ) in phase 2 is between 0.06 and 0.12 h⁻¹.

9. The method according to claim 1, wherein, at the end of Phase 1, approximately 3-10 m·s·/kg of cells are achieved.

10. The method according to claim 1, wherein the yeast is chosen from the group of those capable of consuming xylose or the sugars listed as equivalent glucose.

11. The method according to claim 1, wherein the fed-batch feeding strategy, in at least two phases, increases productivity and reduces propagation time to obtain inocula above 10 g/L (between 15 and 30 h).
